(19) United States
(12) Patent Application Publication
(10) Pub. No.: US 2026/0157724 A1
(43) Pub. Date: Jun. 11, 2026

(54) ULTRASOUND DIAGNOSTIC SYSTEM

(71) Applicant: FUJIFILM Corporation, Tokyo (JP)

(72) Inventors: Shuichi DOKKO, Tokyo (JP); Hiroaki SUGIMOTO, Tokyo (JP)

(73) Assignee: FUJIFILM Corporation, Tokyo (JP)

(21) Appl. No.: 19/383,797

(22) Filed: Nov. 10, 2025

(30) Foreign Application Priority Data
Dec. 5, 2024 (JP) 2024-212122

(51) Int. Cl. A61B 8/12; A61B 8/00

(52) U.S. Cl. CPC A61B 8/12; A61B 8/445; A61B 8/463; A61B 8/5207

(57) ABSTRACT

An ultrasound diagnostic apparatus includes an ultrasound probe and a puncture adapter. The ultrasound probe has a through-hole that guides a biopsy needle at any angle in a predetermined angular range and that has an inverted triangular shape in a head part. The puncture adapter has a needle guide hole that guides the biopsy needle at a fixed angle. The ultrasound diagnostic apparatus generates a second graphic image having a third line representing an inclined angle of the needle guide hole to be superimposed and displayed on an ultrasound image in a case where the puncture adapter is mounted and generates a first graphic image representing a predetermined angular range determined by an inclined angle of an inner wall of the through-hole to be superimposed and displayed on the ultrasound image in a case where the puncture adapter is not mounted.

This application claims priority under 35 USC 119 from Japanese Patent Application No. 2024-212122, filed 5 December, 2024, the disclosure of which is incorporated by reference herein.

### BACKGROUND OF THE INVENTION

### 1. Field of the Invention

The present invention relates to an ultrasound diagnostic system and particularly to display of an ultrasound image.

### 2. Description of the Related Art

An ultrasound diagnostic apparatus acquires biological information on a subject by transmitting ultrasound waves into the subject using an ultrasound probe and receiving reflected waves. The acquired biological information is displayed as an ultrasound image representing a state of the subject. For example, in a case where the ultrasound diagnostic apparatus is used in laparoscopic surgery using a laparoscope, the ultrasound probe is inserted into an abdominal cavity through a trocar disposed in a body wall of the subject.

In the laparoscopic surgery, puncture is performed in some cases. A practitioner inserts a biopsy needle into the abdominal cavity and observes a laparoscopic image to check a position of the biopsy needle. Then, the practitioner causes a distal end of the biopsy needle to reach a target part and then performs collection of a tissue of the target part, injection of a drug into the target part, and the like.

The biopsy needle is guided via a through-hole provided in the ultrasound probe or by using a needle guide prepared separately. In the related art, a puncture adapter that is attachable and detachable to and from an ultrasound probe and that guides a biopsy needle at a desired angle has been proposed (for example, JP2013-233261A).

### SUMMARY OF THE INVENTION

For example, in a case where the puncture adapter that performs puncture at a fixed angle can be attached to the ultrasound probe that can perform puncture at any angle within a predetermined angular range, it is possible to perform both the puncture at any angle and the puncture at the fixed angle by using one ultrasound probe.

Meanwhile, in some cases, the puncture is performed while viewing the ultrasound image of an inside of a body cavity, which is generated and displayed based on a reception signal obtained by the ultrasound probe. In this case, it is convenient to be able to check a puncturable angle that varies depending on whether or not the puncture adapter is mounted while viewing the ultrasound image.

An object of the present disclosure is to display a puncturable angle according to a mounting state of a puncture adapter on an ultrasound probe.

According to an aspect of the present disclosure, there is provided an ultrasound diagnostic system comprising an ultrasound probe that has a distal end part inserted into a body cavity, a puncture adapter that is attachably and detachably mounted on the distal end part, and a processor that is configured to form an ultrasound image based on a reception signal from the ultrasound probe and to generate and display a graphic image superimposed on the ultrasound image, in which the distal end part has a main guide hole that guides a biopsy needle at any angle in a predetermined angular range, the puncture adapter has an insertion part that is inserted into the main guide hole in a case of being mounted on the ultrasound probe and a secondary guide hole that guides the biopsy needle at a fixed angle, and the processor is configured to determine whether or not the puncture adapter in the body cavity is mounted on the distal end part and generate a first graphic image representing the predetermined angular range in a case where the puncture adapter is not mounted on the distal end part and generate a second graphic image representing the fixed angle in a case where the puncture adapter is mounted on the distal end part, as the graphic image.

In addition, the first graphic image may have a first line and a second line that represent an upper limit and a lower limit of the predetermined angular range, respectively.

In addition, the processor may be configured to determine positions and inclined angles of the first line and the second line based on specification information registered in advance.

In addition, the second graphic image may have a third line that represents the fixed angle.

Further, the processor may be configured to determine a position and an inclined angle of the third line based on specification information registered in advance.

Further, as the specification information, position information on an origin in the graphic image and an angle of a reference line that passes through the origin and that is for determining an inclined angle of a line of the graphic image may be set based on a positional relationship with a predetermined position in the distal end part.

In addition, the processor may be configured to determine whether or not the puncture adapter is mounted on the distal end part based on an input by a user.

In addition, a laparoscope may be further included, and the processor may be configured to determine whether or not the puncture adapter is mounted on the distal end part based on a captured image from the laparoscope.

According to the present disclosure, a puncturable angle can be displayed according to a mounting state of the puncture adapter on the ultrasound probe.

### BRIEF DESCRIPTION OF THE DRAWINGS

FIG. 1 is a block diagram showing a schematic configuration of an ultrasound diagnostic apparatus in the present embodiment.

FIG. 2 is an enlarged perspective view showing an example of a head part of a probe in the present embodiment.

FIG. 3 is a perspective view of a head part of the probe in the present embodiment.

FIG. 4 is a perspective view of a puncture adapter in the present embodiment.

FIG. 5 is a side cross-sectional view of the head part of the probe in a state where the puncture adapter is mounted in the present embodiment.

FIG. 6 is a schematic view schematically showing area display in the present embodiment.

FIG. 7 is a schematic view schematically showing line display in the present embodiment.

FIG. 8 is a schematic view schematically showing a modification example of the line display in the present embodiment.

FIG. 9 is a schematic view schematically showing a modification example of the area display in the present embodiment.

FIG. 10 is a view showing an example of an operation panel in the present embodiment.

FIG. 11 is an enlarged view of a portion of the head part in a state where the puncture adapter is not mounted in the present embodiment.

FIG. 12 is an enlarged view of a portion of the head part in a state where the puncture adapter is mounted in the present embodiment.

FIG. 13 is a schematic view schematically showing a modification example of the area display in the present embodiment.

FIG. 14 is a schematic view schematically showing another modification example of the area display in the present embodiment.

### DESCRIPTION OF THE PREFERRED EMBODIMENTS

Hereinafter, an embodiment according to the present disclosure will be described with reference to the drawings.

### Configuration of Ultrasound Diagnostic Apparatus

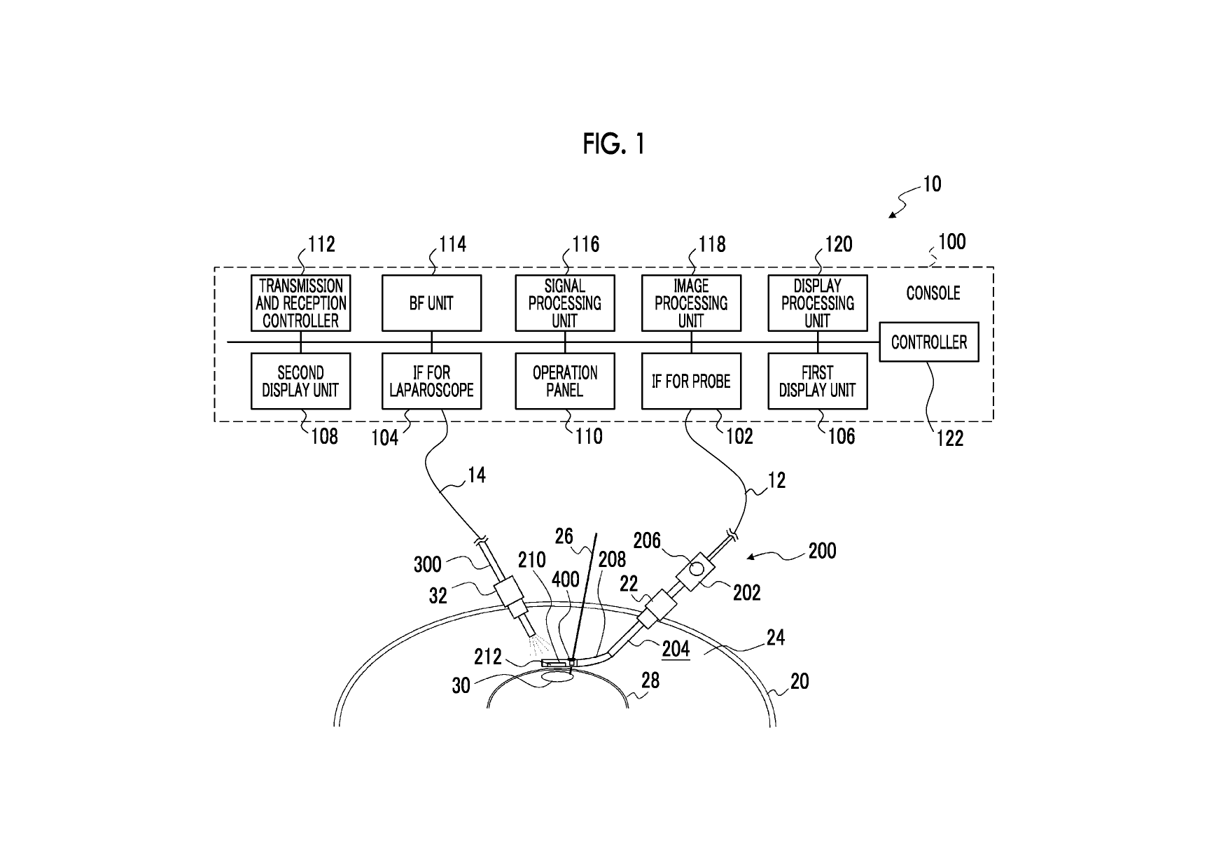

FIG. 1 is a block diagram showing a schematic configuration of an ultrasound diagnostic apparatus 10 in the present embodiment. The ultrasound diagnostic apparatus 10 in the present embodiment has an apparatus main body 100, a probe 200, and a laparoscope 300. The ultrasound diagnostic apparatus 10 can also be referred to as an ultrasound diagnostic system. The ultrasound diagnostic apparatus 10 has a function of executing ultrasound diagnosis using the probe 200 and the laparoscope 300.

The apparatus main body 100 is also called a "console" and has interfaces (IFs) 102 and 104 that connect the probe 200 and the laparoscope 300 to each other, and a first display unit 106, a second display unit 108, and an operation panel 110 as user interfaces. Each of the display units 106 and 108 is a device that displays an image and is configured with, for example, a liquid crystal panel, an organic EL panel, or the like. Among these, the first display unit 106 displays an ultrasound image generated based on an ultrasound signal received by the probe 200. On the other hand, the second display unit 108 displays a captured image from the laparoscope 300. The operation panel 110 is a device operated by an operator (hereinafter, also referred to as a "user") such as a practitioner in order to control input and display of a parameter or the like in ultrasound diagnosis.

The console 100 exerts an ultrasound diagnosis processing function and has a transmission and reception controller 112, a beam forming (BF) unit 114, a signal processing unit 116, an image processing unit 118, a display processing unit 120, and a controller 122.

The transmission and reception controller 112 controls transmission and reception of ultrasound waves caused by each vibration element in the probe 200. This control includes, for example, supply of an electrical transmission signal to each vibration element, amplification of an electrical reception signal from each vibration element, and the like. In the supply of the transmission signal, the transmission and reception controller 112 controls a supply timing of the transmission signal to each vibration element to form a transmission beam of the ultrasound waves.

The beam forming unit 114 performs phasing addition processing on the reception signal from each of the vibration elements in the probe 200. A reception beam is formed by the phasing addition processing. The beam forming unit 114 outputs echo data obtained along the reception beam as a result of the phasing addition processing. In addition, in a case of performing transmission beam forming, the beam forming unit 114 generates a plurality of transmission signals for the transmission beam forming.

The signal processing unit 116 performs various types of signal processing, such as gain correction processing, logarithmic amplification processing, envelope detection processing, and filter processing, on echo data output by the beam forming unit 114.

The image processing unit 118 has a coordinate transformation function and an interpolation function and forms a display frame, that is, an ultrasound image based on a plurality of pieces of beam data output from the signal processing unit 116. The beam data from the signal processing unit 116 is data in a coordinate system of beam scanning, and is configured with a plurality of data points along a direction of a beam corresponding to the beam data. The image processing unit 118 transforms, for example, a signal value of each of the data points of the beam data into a display coordinate system, that is, an ultrasound image coordinate system (generally, an orthogonal coordinate system represented by a set of an x coordinate and a y coordinate). In addition, the image processing unit 118 interpolates a value of a pixel having no value from values of surrounding pixels. The image processing unit 118 forms an ultrasound image, such as a B mode tomographic image, through such coordinate transformation and such interpolation.

The display processing unit 120 forms display screen data by combining an image, text, or the like indicating various types of information on an ultrasound image formed by the image processing unit 118. For example, information to be combined with the ultrasound image includes an ROI indicating a display range of various types of display modes such as a color Doppler mode, a sample volume of a pulse Doppler mode, a line indicating a beam in which the sample volume is positioned, and the like. Then, the display processing unit 120 displays the display screen data formed as described above on the first display unit 106.

The controller 122 controls execution of ultrasound diagnosis processing by controlling an operation of each of components included in the console 100.

Each of the components 102 to 122 in the console 100 is realized by a cooperative operation between a computer mounted on the console 100 and a program that is operated by a CPU mounted on the computer.

The probe 200 is connected to the ultrasound diagnostic apparatus 10 via a cable 12. The probe 200 in the present embodiment is an intracavitary insertion type ultrasound probe. The term "abdominal cavity" is an internal space of a person or the like and is a portion surrounded by an abdominal wall below a diaphragm, more specifically, a space that is surrounded by the abdominal wall and that has a digestive organ such as a stomach and an intestine inside. The term "body cavity" refers to a gap between a body wall and a digestive tract and mainly refers to a thoracic cavity, a pericardial cavity, and the abdominal cavity. In the present embodiment, the term "abdominal cavity" and the term "body cavity" are used synonymously.

The probe 200 mainly comprises a grip part 202 for the operator to grip and an insertion part 204 that extends from the grip part 202 and that is to be inserted into the abdominal cavity. The probe 200 may include the cable 12 described above as a component. An operation part 206 for the operator to operate is provided in the grip part 202. A trocar (guide tube) 22 that passes through an abdomen (hereinafter, referred to as an “abdominal wall”) 20 of the body wall for the probe 200 is attached to the abdominal wall 20, and the insertion part 204 of the probe 200 is inserted into an abdominal cavity 24 from an internal passage of the trocar 22. In a case where an inner diameter of the trocar 22 is approximately 12 mm, an outer diameter of the insertion part 204 of the probe 200 needs to be smaller than the inner diameter of the trocar 22 and is, for example, approximately 10 mm.

The insertion part 204 has a bending part 208 that bends up, down, left, or right in response to an operation of the operation part 206 and a distal end part (hereinafter, referred to as a “head part”) 210 on a distal end side extending from the bending part 208. A transducer array 212 is disposed in the head part 210 in a longitudinal direction. The transducer array 212 corresponds to “each vibration element” described above. The ultrasound diagnostic apparatus 10 performs ultrasound diagnosis inside the body cavity by causing the transducer array 212 to emit ultrasound waves. The operator bends the bending part 208 by operating the operation part 206, thereby securing a diagnosis visual field. A through-hole for guiding a biopsy needle is provided on a bending part 208 side of the head part 210. A biopsy needle guiding cutout that guides the biopsy needle may be provided on the distal end side of the head part 210.

Then, a biopsy needle guiding adapter (hereinafter, simply referred to as a "puncture adapter") 400 is attached at a position where the through-hole is disposed. A biopsy needle 26 is inserted from the abdominal wall 20. A distal end portion of the biopsy needle 26 passes through a needle guide hole provided in the puncture adapter 400 and reaches a target part, for example, a tumor or the like 30 of an organ 28.

In addition, a trocar 32 for inserting the laparoscope 300 into the abdominal cavity 24 is attached to the abdominal wall 20. A light source (not shown) is provided at a distal end portion of the laparoscope 300, and the laparoscope 300 images an irradiation range in the light source, for example, a range including the head part 210 of the probe 200 by being operated by the user.

The configuration of the ultrasound diagnostic apparatus 10 and a relationship between a human body to be subjected to ultrasound diagnosis and the ultrasound diagnostic apparatus 10 have been described above with reference to FIG. 1. The ultrasound diagnostic apparatus 10 in the present embodiment may have basically the same hardware configuration as in the related art.

### Structures of Probe 200 and Puncture Adapter 400

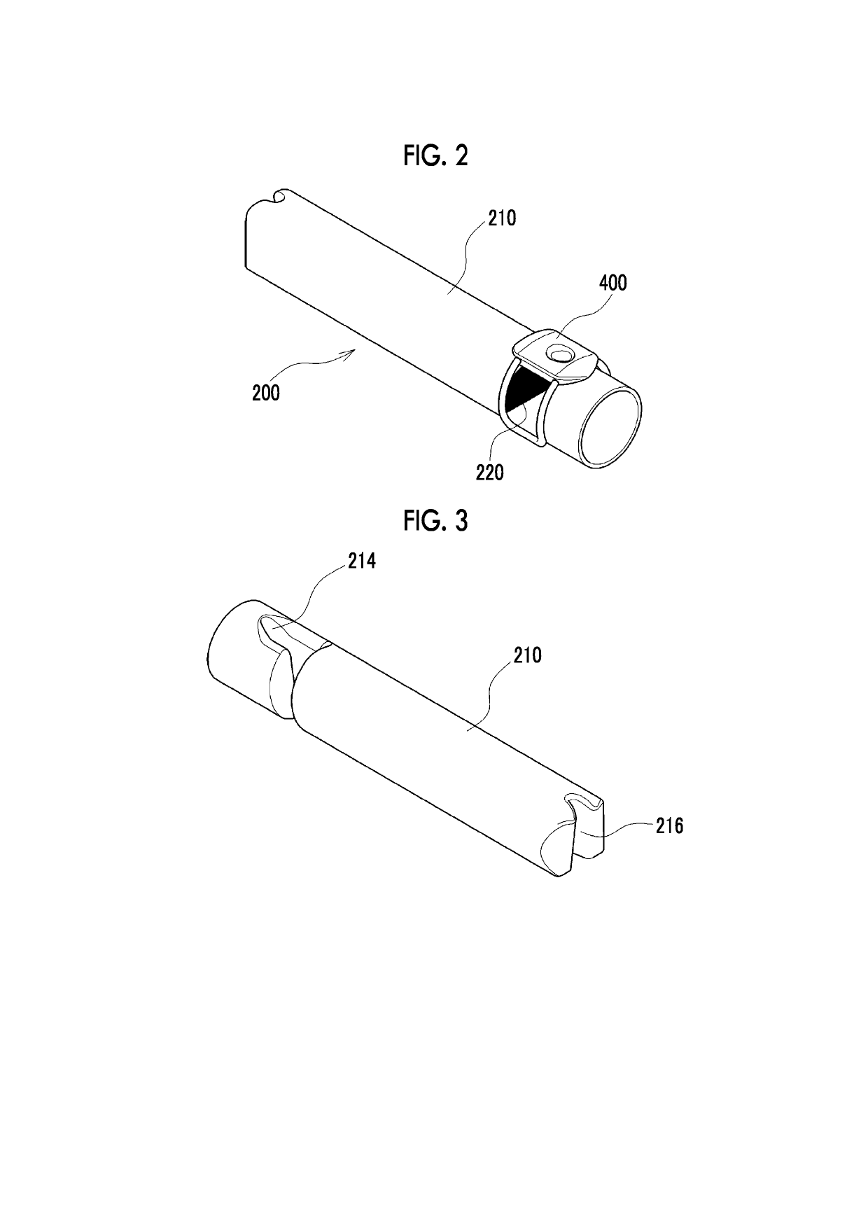

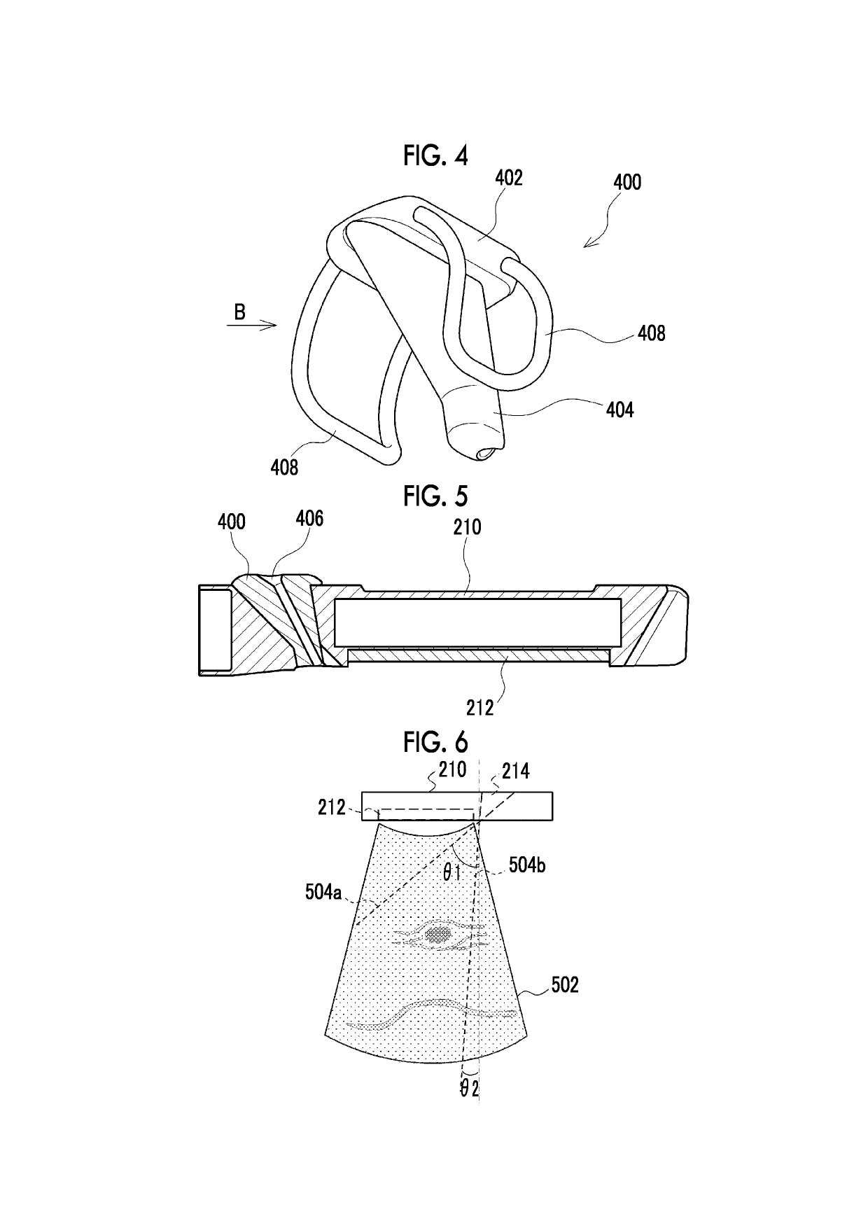

FIG. 2 is an enlarged perspective view showing an example of the head part 210 of the probe 200 shown in FIG. 1. As shown in FIG. 2, the puncture adapter 400 can be mounted on the head part 210. FIG. 3 is a perspective view of the head part 210 in a state where the puncture adapter 400 is not mounted. FIG. 2 is a perspective view in which a right side surface of the head part 210 is viewed from a direction of the bending part 208, while FIG. 3 is a perspective view in which a left side surface of the head part 210 is viewed from a direction of the distal end of the head part 210. FIG. 4 is a perspective view of the puncture adapter 400 in the present embodiment. The puncture adapter 400 shown in FIG. 4 is a perspective view in an arrow B direction, that is, in a case of being viewed from the bending part 208 side in a case of being mounted on the probe 200. FIG. 5 is a side cross-sectional view in a case where the puncture adapter 400 is mounted on the head part 210. Although components such as a circuit for transmitting and receiving an ultrasound signal may be incorporated inside the head part 210, the components are not shown in FIG. 5. Hereinafter, an engagement relationship between the probe 200 and the puncture adapter 400 will be described with reference to FIGS. 1 to 5.

The transducer array 212 and a through-hole 214 for guiding the biopsy needle are disposed in the head part 210, that is, a distal end part of the probe 200. In the probe 200 in the present embodiment, the through-hole 214 is provided on a bending part side of the transducer array 212. The through-hole 214 may be disposed on the distal end side of the transducer array 212. A biopsy needle guiding cutout 216 is formed in the distal end of the head part 210 in the present embodiment. By increasing a cutout amount of the biopsy needle guiding cutout 216 from an upper surface toward a lower surface of the head part 210, the biopsy needle guiding cutout 216 can guide a distal end of the biopsy needle 26 to enter below the transducer array 212.

A cross-sectional shape of a needle entrance portion of the through-hole 214 is an inverted triangular shape. That is, the through-hole 214 has a tapered shape from the needle entrance portion toward a needle exit portion. Accordingly, the distal end of the biopsy needle inserted into the through-hole 214 can be guided to enter below the transducer array 212. In a state where the puncture adapter 400 is not mounted on the probe 200, the through-hole 214 has such a shape, so that the puncture can be performed within a range of a predetermined angle. In the following description, the puncture in which the biopsy needle is guided at any angle within a predetermined angular range will be referred to as "area puncture".

A mark 220 having an inverted triangular shape is attached to at least one side surface of the head part 210 at a position corresponding to the through-hole 214. The mark 220 is a flag for guiding the mounting of the puncture adapter 400. The mark 220 is printed in an inverted triangular shape in accordance with the shape of the through-hole 214. Accordingly, even in a case where the needle entrance portion of the through-hole 214 is not visible on the second display unit 108, the user can know the position and the size of the needle entrance portion of the through-hole 214 from the mark 220.

The structure of the puncture adapter 400 can be roughly divided into an adapter body and a clip mechanism. The adapter body has a body part 402 and an insertion part 404 and further has a needle guide hole 406 that communicates with the body part 402 and the insertion part 404.

The body part 402 is formed in a thin plate shape and is disposed to cover a needle entrance portion of the through-hole 214 of the probe 200 in a case of being mounted on the probe 200. The body part 402 is formed in a so-called streamlined shape such that thicknesses of both end parts of the probe 200 in an insertion and removal direction from the trocar 22 are reduced in order to perform smooth insertion and removal from the trocar 22 in a state of being mounted on the probe 200.

In a case where the puncture adapter 400 is mounted on the head part 210, the insertion part 404 is inserted into the through-hole 214 formed in the probe 200. The insertion part 404 is formed in an inverted triangular shape in accordance with the shape of the through-hole 214 formed in the probe 200.

The needle guide hole 406 linearly penetrates from the needle entrance portion provided in the upper surface of the body part 402 to the needle exit portion provided in the tapered distal end part of the insertion part 404, and guides the biopsy needle 26 inserted from the body part 402 to the target part (in the case of the present embodiment, the tumor or the like 30).

In the probe 200 in a state where the puncture adapter 400 is mounted, the puncture is possible at a fixed angle. In the following description, the puncture in which the biopsy needle is guided at a predetermined fixed angle will be referred to as "line puncture".

According to the present embodiment, the area puncture can be performed with the probe 200 in a state where the puncture adapter 400 is not mounted. Since the puncture adapter 400 in the present embodiment has a shape that enables being mounted on the through-hole 214 in which the area puncture is possible, the line puncture can be performed with the probe 200 in a state where the puncture adapter 400 is mounted. That is, the user can perform the area puncture or the line puncture using the same probe 200 by selecting whether or not to mount the puncture adapter 400 to the probe 200 outside the abdominal cavity.

On the other hand, the clip mechanism is formed of a pair of clips 408 that extends from a side surface of the body part 402 in opposite directions and that is curved along the outer surface of the probe 200. A root portion of the pair of clips 408, that is, a portion that is bonded to the body part 402 is formed of an elastic member such as spring steel. The clips 408 are formed in a curved shape along the outer surface of the mounting position of the probe 200 in order to make it difficult for the clips 408 to be caught in a case where the insertion part 204 of the probe 200 is inserted into and removed from the trocar 22.

In a case of performing the area puncture, the user does not attach the puncture adapter 400 to the probe 200. On the other hand, in a case of performing the line puncture, the user attaches the puncture adapter 400 to the probe 200 outside the body cavity. That is, the user aligns the puncture adapter 400 with the position of the through-hole 214 of the head part 210, pushes the insertion part 404 into the through-hole 214 until the insertion part 404 is fitted into the through-hole 214, the body part 402 touches the head part 210, and the clips 408 are firmly gripped. Accordingly, the puncture adapter 400 is reliably mounted such that the puncture adapter 400 does not come off even in a case where the probe 200 is inserted into and removed from the trocar 22.

### Display of Ultrasound Image

As described above, in the present embodiment, the puncture adapter 400 can be easily attached to the probe 200 outside the body cavity, and an attachment state of the puncture adapter 400 in the body cavity can be easily checked. In addition, smooth insertion and removal of the probe 200 from the trocar 22 passing through the abdominal wall 20 can be performed. In the present embodiment, the area puncture can be performed in a case where the puncture adapter 400 is not mounted on the probe 200, and the line puncture can be performed by mounting the puncture adapter 400 on the through-hole 214 of the probe 200. However, both the area puncture and the line puncture using the same probe 200 can be performed. However, in a case of performing puncture, it is convenient in a case where a puncturable angle that varies depending on whether or not the puncture adapter 400 is mounted can be checked while viewing an ultrasound image displayed on the first display unit 106.

Therefore, in the present embodiment, the puncturable angle is displayed according to a mounting state of the puncture adapter 400 on the probe 200. Specifically, in a case where the puncture adapter 400 is not mounted on the probe 200, a predetermined range of angles at which the area puncture is possible is displayed, and in a case where the puncture adapter 400 is mounted on the probe 200, a fixed angle at which the line puncture is performed is displayed.

The display is performed by the display processing unit 120 under the control of the controller 122. That is, the display processing unit 120 forms an ultrasound image based on a reception signal from the probe 200 to display the ultrasound image on the first display unit 106, but generates a graphic image to be superimposed and displayed on the ultrasound image. Specifically, in a case where the puncture adapter 400 is not mounted on the head part 210 of the probe 200, the display processing unit 120 generates a first graphic image that represents the predetermined angular range, that is, a range of an angle at which the area puncture is possible, as the graphic image. In the following description, display of the generated first graphic image in a superimposed manner on the ultrasound image is also referred to as "area display".

On the other hand, in a case where the puncture adapter 400 is mounted on the head part 210 of the probe 200, the display processing unit 120 generates a second graphic image that represents the predetermined fixed angle, that is, an angle at which the line puncture is possible, as the graphic image. In the following description, display of the generated second graphic image in a superimposed manner on the ultrasound image is also referred to as "line display".

In the following description, display of the first graphic image or the second graphic image in a superimposed manner on the ultrasound image will be referred to as "guide display". The guide display can also be referred to as a general term for the area display and the line display.

Hereinafter, the area display and the line display, which are characteristic in the present embodiment, will be described in detail with reference to the drawings.

FIG. 6 is a schematic view schematically showing area display in the present embodiment. FIG. 6 shows a screen display example in a case where the first graphic image is superimposed and displayed on an ultrasound image 502 in a B mode displayed on the first display unit 106. Further, for convenience of description, FIG. 6 shows a positional relationship between the ultrasound image 502 and the head part 210 of the probe, particularly, a positional relationship between the transducer array 212 and the through-hole 214 of the head part 210. The first graphic image includes a first line 504a and a second line 504b that represent an upper limit and a lower limit of the predetermined angular range, respectively. In a case where a direction orthogonal to the transducer array 212, that is, a vertical direction in FIG. 6, which is shown by a one-dot chain line in FIG. 6, is set to 0 degrees, an inclination (hereinafter, referred to as an “inclined angle”) of the first line 504a is θ1, and an inclined angle of the second line 504b is θ2. This angle depends on an angle of an inner wall of the through-hole 214 which is a main guide hole for guiding the biopsy needle.

As shown in FIG. 6, in a case where the puncture adapter is not mounted on the through-hole 214, the predetermined angular range in which the puncture is possible by the first line 504a and the second line 504b is displayed in a superimposed manner on the ultrasound image 502. Therefore, the user can perform the puncture with reference to the displayed angular range.

In FIG. 6, an example in which the first line 504a is displayed in the ultrasound image 502 and the second line 504b is displayed by being extended to the outside of the ultrasound image 502 is shown, but display ranges of the respective lines 504a and 504b, that is, a relationship between the display range of the ultrasound image 502 and the display range of the first graphic image may be determined as appropriate. The same applies to the line display to be described later.

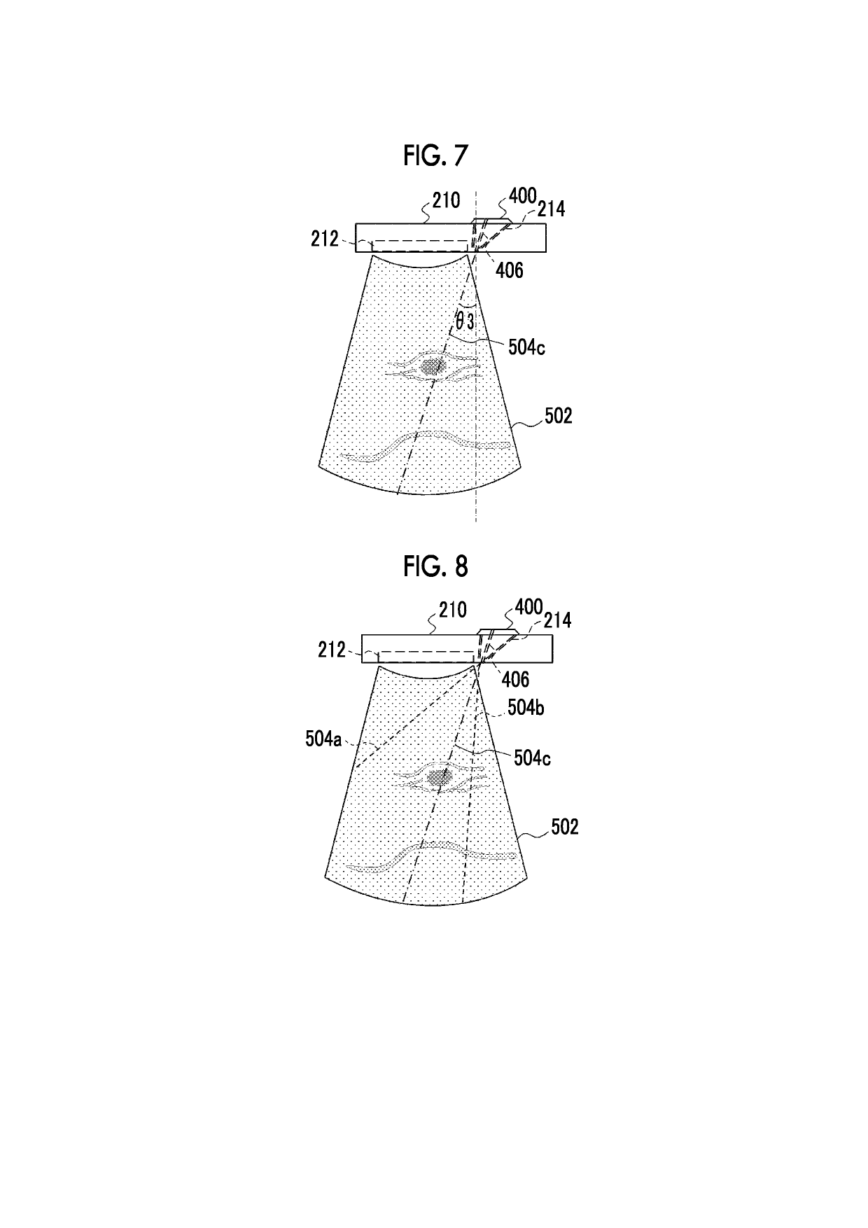

FIG. 7 is a schematic view schematically showing line display in the present embodiment. FIG. 7 shows a screen display example in a case where the second graphic image is superimposed and displayed on the ultrasound image 502 in the B mode displayed on the first display unit 106. Further, for convenience of description, FIG. 7 shows a positional relationship between the ultrasound image 502 and the head part 210 of the probe, particularly, a positional relationship between the transducer array 212 of the head part 210 and the puncture adapter 400 having the insertion part inserted into the through-hole 214. The second graphic image includes a third line 504c that represents the predetermined fixed angle. In a case where the direction orthogonal to the transducer array 212, that is, a vertical direction in FIG. 7, which is shown by a one-dot chain line in FIG. 7, is set to 0 degrees, an inclination of the third line 504c is θ3. The inclined angle θ3 depends on the inclined angle of the needle guide hole 406 which is a secondary guide hole formed in the puncture adapter 400 that guides the biopsy needle.

As shown in FIG. 7, in a case where the puncture adapter 400 is mounted on the through-hole 214 of the head part 210, the predetermined angle at which the puncture is possible by the third line 504c in a superimposed manner on the ultrasound image 502. Therefore, the user can perform the puncture such that the biopsy needle is not detached from the third line 504c.

As described above, FIG. 6 shows a display example of the area display, and FIG. 7 shows a display example of the line display. However, it is not required for content to be displayed to be limited to this example. For example, FIG. 8 shows a modification example of the line display.

In FIG. 8, since the puncture adapter 400 is mounted on the through-hole 214 of the head part 210, the line display shown in FIG. 7 is basically performed. However, as shown in FIG. 8, both the first line 504a and the second line 504b to be displayed in the area display may be displayed. In this case, the second graphic image includes the first line 504a and the second line 504b in addition to the third line 504c. Alternatively, the first graphic image may be superimposed and displayed on the ultrasound image 502 in addition to the second graphic image. Each of the lines 504a to 504c can be drawn with reference to specification information to be described later.

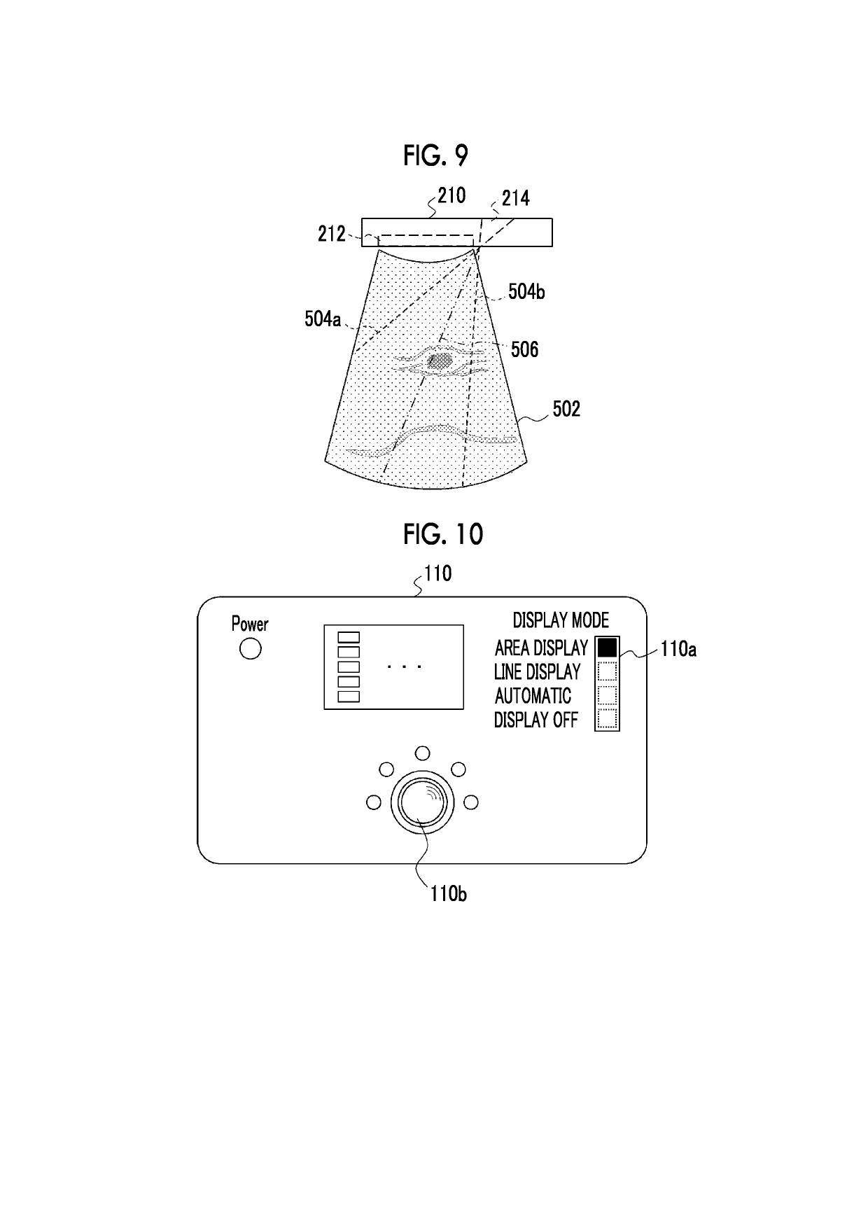

In FIG. 9, since the puncture adapter 400 is not mounted on the through-hole 214 of the head part 210, the area display shown in FIG. 6 is basically performed. However, it can be assumed that the user wants to identify beforehand at which angle the puncture is actually performed, even though it can be seen that it is possible to perform the puncture between the first line 504a and the second line 504b. Therefore, in the present embodiment, a fourth line 506 can be displayed in response to an instruction from the user. The user operates the operation panel 110 to input the display and a display angle of the fourth line 506. The display processing unit 120 may generate the first graphic image to include the fourth line 506 in response to the user instruction or may generate a third graphic image having the fourth line 506 and superimpose and display the first graphic image and the third graphic image on the ultrasound image 502.

Meanwhile, the controller 122 in the present embodiment determines whether or not the puncture adapter 400 is mounted on the probe 200 and selects a display mode, that is, any one of the area display or the line display. Herein, a method of determining whether or not the puncture adapter 400 is mounted will be described.

FIG. 10 is a view showing an example of the operation panel 110 in the present embodiment. The operation panel 110 is provided with a switch 110a for selecting a display mode. In addition, a trackball 110b for the user to give an instruction on the angle of the fourth line 506 shown in FIG. 9 is provided. In a case where the user mounts the puncture adapter 400 on the probe 200, the user operates the switch 110a to select the line display. In addition, in a case where the puncture adapter 400 is not mounted on the probe 200, the user operates the switch 110a to select the area display. FIG. 6 shows a case where the area display is selected. The controller 122 determines whether or not the puncture adapter 400 is mounted on the probe 200 based on the input instruction from the user.

In addition, in a case where the user operates the switch 110a to select "display off", the controller 122 does not instruct the display processing unit 120 to generate a graphic image. Accordingly, each of the lines 504a to 504c shown in FIGS. 6 to 8 is not displayed on the ultrasound image.

In addition, in a case where the user operates the switch 110a to select "automatic", the controller 122 determines whether or not the puncture adapter 400 is mounted on the head part 210 in person, instead of the selection operation by the user. In the case of the present embodiment, since the laparoscope 300 is connected to the ultrasound diagnostic apparatus 10, the controller 122 may perform the determination based on a captured image from the laparoscope 300.

For example, in a case where the console 100 acquires a captured image from the laparoscope 300, the controller 122 analyzes the captured image to extract the head part 210 and determines whether or not the puncture adapter 400 is mounted on the extracted head part 210. For example, an image of the head part 210 on which the puncture adapter 400 is not mounted is acquired beforehand as a reference image. Since it is not clear from which angle the laparoscope 300 images the head part 210, it is desirable that the reference image is a three-dimensional image. Alternatively, a plurality of two-dimensional images may be prepared.

In any case, the controller 122 collates an image of the head part 210 extracted from a captured image with the reference image. In a case where it is determined that the image of the head part 210 being imaged is the same as the reference image as a result of the collation, the controller 122 determines that the puncture adapter 400 is not mounted. In this case, the controller 122 selects the area display and instructs the display processing unit 120 to generate the first graphic image. On the other hand, in a case where it is determined that the image of the head part 210 being imaged is not the same as the reference image, the controller 122 determines that the puncture adapter 400 is mounted. In this case, the controller 122 selects the line display and instructs the display processing unit 120 to generate the second graphic image.

Automatic determination based on a captured image from the laparoscope 300 is an example, and the present invention is not limited thereto. For example, the image of the head part 210 on which the puncture adapter 400 is mounted may be used as the reference image, or both images may be used as the reference images.

Although it has been described that whether or not the puncture adapter 400 described above is mounted is determined by the controller 122, the mounting may be performed in cooperation with other components. For example, the controller 122 may instruct the image processing unit 118 to perform image analysis on a captured image.

In addition, a learning model generated by performing machine learning with an image of the head part 210 on which the puncture adapter 400 is mounted and an image of the head part 210 on which the puncture adapter 400 is not mounted as teacher images may be used. The learning model does not necessarily need to be executed on the console 100 and may be generated by using another computer. In any case, in a case where the learning model is used, the learning model is registered beforehand in the console 100. Then, in a case where the console 100 acquires a captured image from the laparoscope 300, the controller 122 inputs the captured image into the learning model to adopt an output of the learning model, that is, whether or not the puncture adapter 400 is mounted as a determination result.

In the present embodiment, the user can select the area display or the line display in person and can further select the automatic determination on whether or not the puncture adapter 400 is mounted on the head part 210. However, the method of determining whether or not the puncture adapter 400 is mounted on the head part 210 is an example, and another method may be used. In addition, in the present embodiment, the user can select a display mode in person by operating the switch 110a and can further select the automatic determination of the display mode, but may be configured to adopt only one of the two.

As described above, according to the present embodiment, content of the guide display, that is, the area display or the line display is discriminated according to whether or not the puncture adapter 400 is mounted on the head part 210. The user may perform the puncture with reference to the lines 504a to 504c displayed by the guide display.

However, the lines 504a to 504c may not always be correctly displayed. For example, the head part 210 of the probe 200 or the puncture adapter 400 is manufactured according to a design specification (hereinafter, referred to as "design information"), but there is also a possibility in which an error occurs between the design information and the actual product as a result of the manufacturing. Therefore, in the present embodiment, a unit that can eliminate such an error is provided. Correction for eliminating this error will be described below.

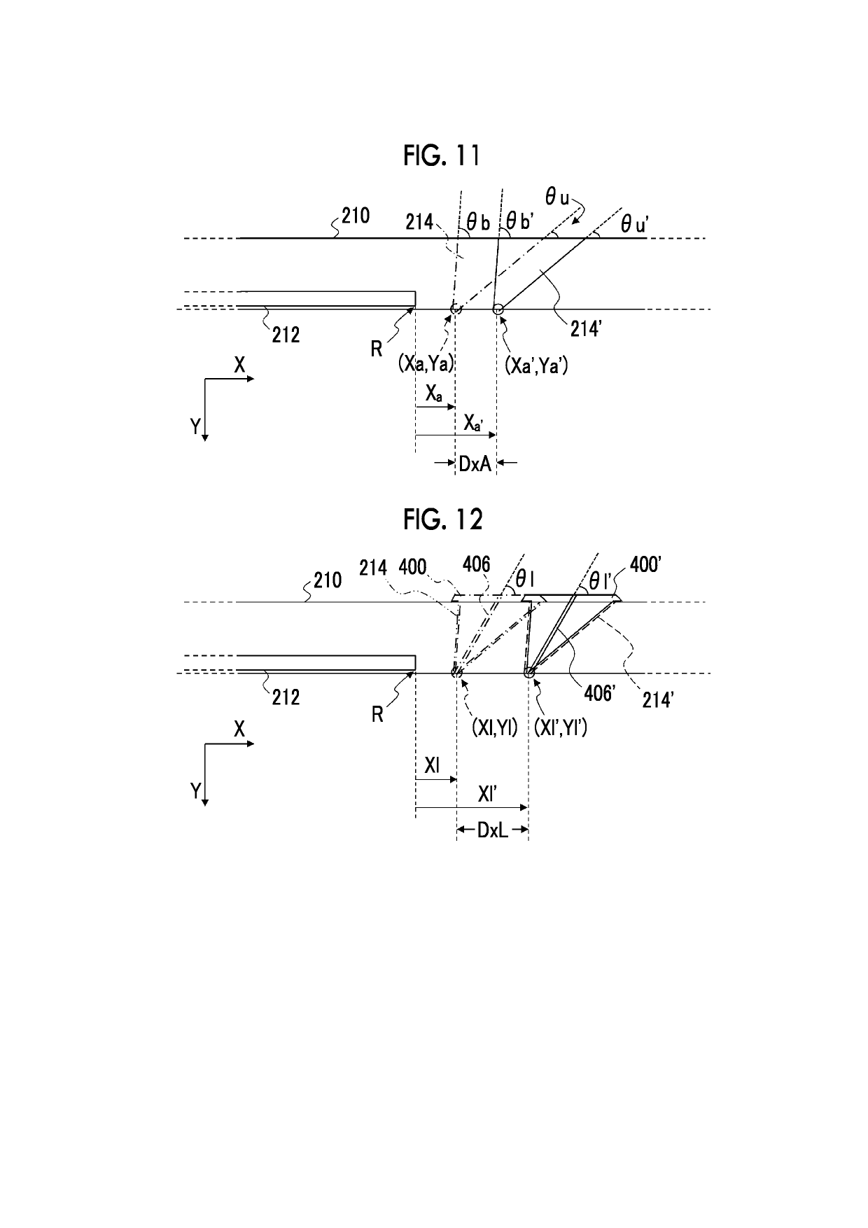

FIG. 11 is an enlarged view of a portion of the head part 210 in a state where the puncture adapter is not mounted. FIG. 11 shows the transducer array 212 and the through-hole 214. Since the puncture adapter 400 is not mounted on the through-hole 214, it can be seen that this is an example in a case of the area display. In FIG. 11, the through-hole 214 shown by a one-dot chain line indicates a reference position to be formed according to design information, and a through-hole 214' shown by a solid line indicates a through-hole formed at a position deviated from the reference position in practice, although the through-hole 214' is formed according to the design information.

In the area puncture, the biopsy needle always passes through an exit of the through-hole 214 regardless of an angle at which the puncture is performed. Therefore, in the present embodiment, the exit of the through-hole 214 is defined as an origin of a two-dimensional coordinate system in the first graphic image.

In the two-dimensional coordinate system, a direction along the transducer array 212 is an X-axis direction, and a direction orthogonal to the transducer array 212 is a Y-axis direction. In a case where the coordinates of the origin in the through-hole 214 are (Xa, Ya) and the coordinates of the origin in the through-hole 214' are (Xa', Ya'), it is shown that an error of DxA (= Xa' - Xa) occurs in the X-axis direction in the actual product.

In the area display, the first graphic image representing a puncture angle range by the through-hole 214 is superimposed and displayed on the ultrasound image generated based on transmission and reception by the transducer array 212. Therefore, in a case where a predetermined position in the transducer array 212 is set as a reference point R and a relative position from the reference point R to the exit of the through-hole 214 is known, the origin in the first graphic image can be identified. In FIG. 11, a corner of the transducer array 212 close to the through-hole 214 is set as the reference point R. The position (Xa, Ya) of the relative origin from the reference point R can be identified from the design information on the probe 200. In FIG. 11, an error DxA (= Xa' - Xa) actually occurs, but an X-axis position Xa' (= Xa + DxA) of the actual origin can be corrected. Although not shown, correction can be made on the Y-axis direction in the same manner as the X-axis direction.

In addition, a predetermined position in the head part 210 may be used as a reference point. However, in consideration of the fact that an error occurs in the mounting position of the transducer array 212 in the head part 210, it is preferable to set any position in the transducer array 212 as a reference point.

As described above, in a case where the actual position of the through-hole 214' can be identified, the display processing unit 120 draws each of the lines 504a and 504b at a predetermined angle with the origin (Xa', Ya') as a start point.

The position of the origin can be corrected as described above, but an error can occur also in the inclined angle of the inner wall of the through-hole 214 for determining the inclined angles θ1 and θ2 of the lines 504a and 504b, respectively. In addition, the inclined angle θ1 of the first line 504a is determined by an inclined angle θu of the through-hole 214. The inclined angle θ2 of the second line 504b is determined by the inclined angle θb of the through-hole 214. As described above, the inclined angle of the inner wall of the through-hole 214' actually formed can be an angle θu' (≠ θu) or an angle θb' (≠ θb) due to the error that occurs in a manufacturing process. Therefore, the angles θu and θb of the inner wall of the through-hole 214 are corrected to θu' and θb'.

The origin (Xa', Ya') and the angles θu' and θb' of the actual through-hole 214' described above are found in inspection before shipment with respect to the probe 200. Therefore, the origin (Xa, Ya) and the angles θu and θb in the design are corrected as described above to create the specification information. As this specification information, in a case where position information on the origin in the first graphic image, that is, coordinate data of (Xa', Ya') and two reference lines, including first and second reference lines passing through the origin are assumed based on a positional relationship with a predetermined position (the "reference point R") in the head part 210, the inclined angle θu' of the first reference line and the inclined angle θb' of the second reference line are set. The specification information is stored in the ultrasound diagnostic apparatus 10 to which the probe 200 is connected.

The display processing unit 120 generates the first graphic image based on the specification information. That is, the display processing unit 120 draws the first line 504a with the origin (Xa', Ya') as the start point and the inclined angle θu' of the first reference line as θ1. In addition, the display processing unit 120 draws the second line 504b with the origin (Xa', Ya') as the start point and the inclined angle θb' of the second reference line as θ2.

According to the present embodiment, the first line 504a and the second line 504b can be drawn based on the specification information on the probe 200 that is actually used, instead of the design information. Accordingly, the user can perform the puncture within a predetermined range that coincides with the probe 200 to be actually used.

FIG. 12 is an enlarged view of a portion of the head part 210 in a state where a puncture adapter 400' is mounted. FIG. 12 shows the puncture adapter 400 in addition to the transducer array 212 and the through-hole 214. Since the puncture adapter 400 is mounted on the through-hole 214, it can be seen that this is an example of a case of the line display. In FIG. 12, the through-hole 214 shown by a two-dot chain line indicates a reference position to be formed according to the design information, and the puncture adapter 400 shown by a one-dot chain line indicates the puncture adapter mounted on the through-hole 214. On the other hand, the through-hole 214' shown by a broken line indicates a through-hole that is actually formed at a position deviated from the reference position according to the design information. The puncture adapter 400' shown by a solid line indicates the puncture adapter that is actually mounted on the through-hole 214'.

In the line puncture, the biopsy needle always passes through an exit of the needle guide hole 406 of the puncture adapter 400. Therefore, in the present embodiment, the exit of the needle guide hole 406 is defined as an origin of a two-dimensional coordinate system in the second graphic image.

A basic idea of position correction in the line display is the same as that described in the area display. In a case where the coordinates of the origin in the exit of the needle guide hole 406 in the design are (Xl, Yl) and the coordinates of the origin in the exit of the needle guide hole 406' are (Xl', Yl'), it is shown that an error of DxL (= Xl' - Xl) occurs in the X-axis direction in the actual product. Therefore, an X-axis position Xl' (= Xl + DxL) of the actual origin can be corrected. Although not shown, correction can be made on the Y-axis direction in the same manner as the X-axis direction.

In addition, an error can occur in an inclined angle θl of the needle guide hole 406 for determining the inclined angle θ3 of the third line 504c. Therefore, the inclined angle θl of the needle guide hole 406 is corrected to θl'.

The exit of the needle guide hole 406' in the actual puncture adapter 400', that is, the origin point (Xl', Yl') and the angle θl' are found in inspection by mounting the actually manufactured puncture adapter 400' on the through-hole 214' of the head part 210 before shipment. Therefore, the origin (Xl, Yl) and the angle θl in the design are corrected as described above to create the specification information. In the specification information, the position information on the origin in the second graphic image, that is, the coordinate data of (Xl', Yl') and the inclined angle θl' in one third reference line passing through the origin are set based on a positional relationship with the predetermined position (the "reference point") in the head part 210. The specification information is stored in the ultrasound diagnostic apparatus 10 to which the probe 200 is connected.

The display processing unit 120 generates the second graphic image based on the specification information. That is, the display processing unit 120 draws the third line 504c with the origin (Xl', Yl') as the start point and the inclined angle θl' of the third reference line as θ3.

According to the present embodiment, the third line 504c can be drawn based on the specification of the probe 200 that is actually used, instead of the design information. Accordingly, the user can perform the puncture at the predetermined fixed angle that coincides with the probe 200 to be actually used.

In the present embodiment, the specification information including the coordinate data of the origin after correction and the value of the inclined angle is transmitted to the console 100. However, the specification information including a correction value, that is, the above-described DxA, DxL, an inclined angle error, and the like may be created and stored in the console 100 together with the design information. In this case, the display processing unit 120 displays each of the lines 504a to 504c after correcting the origin and the inclined angle in a case of generating a graphic image.

Meanwhile, in the case of the area puncture, the user performs the puncture with the inclined angle of the first line 504a as an upper limit and the inclined angle of the second line 504b as a lower limit with reference to the area display shown in FIG. 6. In a case of performing the area display, the area display shown in FIG. 6 may be displayed in a different display form for convenience of the user. For example, in the ultrasound image, the display form is different between a range surrounded by the first line 504a and the second line 504b and outside the range.

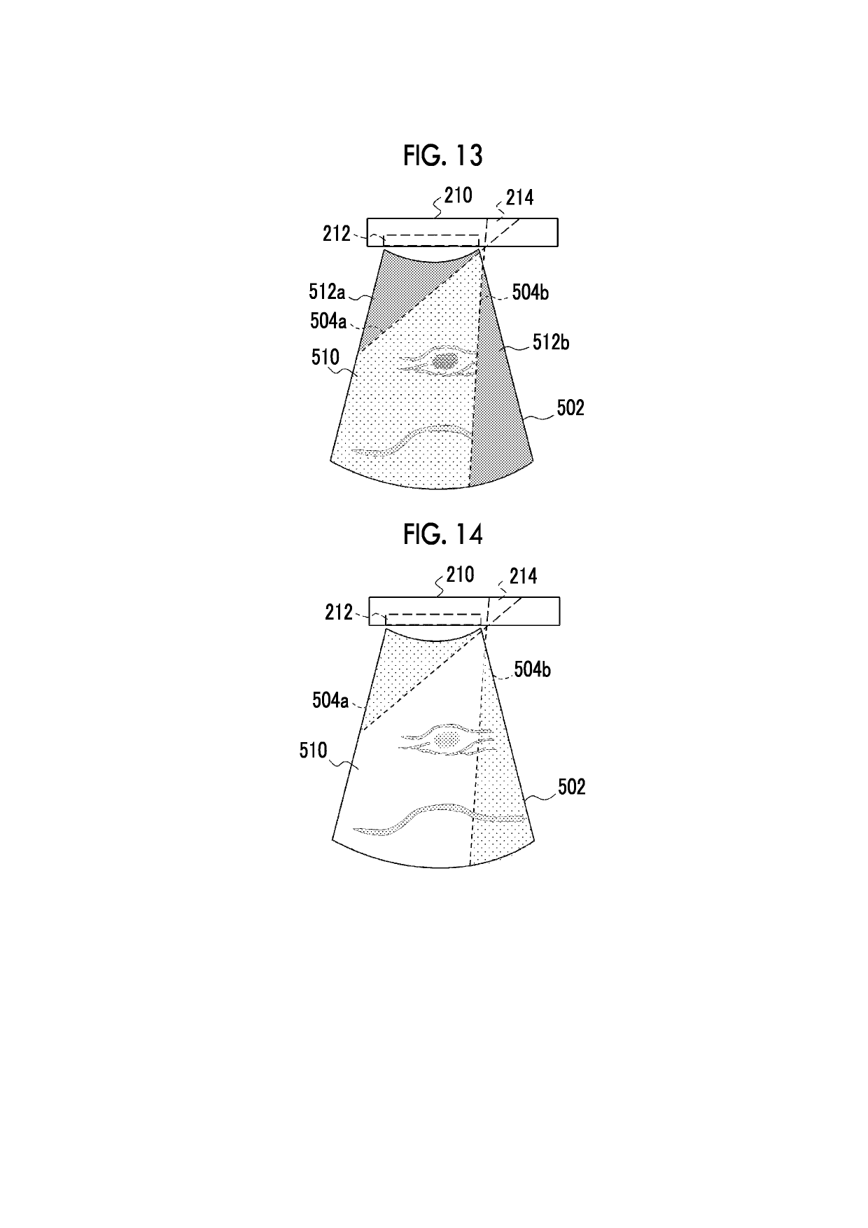

FIG. 13 is a schematic view schematically showing a modification example of the area display in the present embodiment. In FIG. 13, the inside of the range surrounded by the first line 504a and the second line 504b is displayed as usual, that is, in the same manner as in FIG. 6, while the outside thereof is displayed to be difficult to be visible or not to be visible. For example, brightness of an outer image region is decreased to display the outer image region in a dark manner.

FIG. 14 is a schematic view schematically showing another modification example of the area display in the present embodiment. In FIG. 14, the outside of the range surrounded by the first line 504a and the second line 504b is displayed as usual, while the inside of the range thereof is displayed to be easily visible or to be easily noticed. For example, brightness of an inner image region is increased to display the inner image region in a bright manner or to display the inner image region in a bright background color.

In this manner, by making a display form different between a range in which the area puncture is possible and a range in which the area puncture is not possible, the user may display a puncturable range to enhance visibility of the puncturable range.

According to the present embodiment, the area display or the line display can be performed according to whether or not the puncture adapter 400 is mounted. In the area display, an angular range at which the area puncture is possible is displayed by the lines 504a and 504b. In the line display, an angle at which the line puncture is possible is displayed by the third line 504c. In FIG. 6, each of the lines 504a and 504b is shown by broken lines, in FIG. 7, the third line 504c is shown by a one-dot chain line, and in FIG. 9, the fourth line 506 is shown by a two-dot chain line. The display examples show that it is not necessary to display the lines in the same line type, and the line types may be different from each other. In addition, the lines 504a to 504c and 506 may be displayed in various display forms, such as by changing a display color or continuously displaying a symbol such as “+” to form the lines, without being limited to the line type. In the graphic image, a line type other than the line types used in the ultrasound image may be used such that it is easy to discriminate whether the line displayed on the screen is the line displayed in the ultrasound image or the line displayed in the graphic image.

What is claimed is:

1. An ultrasound diagnostic system comprising:
an ultrasound probe that has a distal end part inserted into a body cavity;
a puncture adapter that is attachably and detachably mounted on the distal end part; and
a processor that is configured to form an ultrasound image based on a reception signal from the ultrasound probe and to generate and display a graphic image superimposed on the ultrasound image,
wherein the distal end part has a main guide hole that guides a biopsy needle at any angle in a predetermined angular range,
the puncture adapter has
an insertion part that is inserted into the main guide hole in a case of being mounted on the ultrasound probe, and
a secondary guide hole that guides the biopsy needle at a fixed angle, and
the processor is configured to:
determine whether or not the puncture adapter in the body cavity is mounted on the distal end part; and
generate a first graphic image representing the predetermined angular range in a case where the puncture adapter is not mounted on the distal end part and generate a second graphic image representing the fixed angle in a case where the puncture adapter is mounted on the distal end part, as the graphic image.

2. The ultrasound diagnostic system according to claim 1,
wherein the first graphic image has a first line and a second line that represent an upper limit and a lower limit of the predetermined angular range, respectively.

3. The ultrasound diagnostic system according to claim 2,
wherein the processor is configured to:
determine positions and inclined angles of the first line and the second line based on specification information registered in advance.

4. The ultrasound diagnostic system according to claim 3,
wherein position information on an origin in the graphic image and an angle of a reference line that passes through the origin and that is for determining an inclined angle of a line of the graphic image is set in the specification information based on a positional relationship with a predetermined position in the distal end part.

5. The ultrasound diagnostic system according to claim 1,
wherein the second graphic image has a third line that represents the fixed angle.

6. The ultrasound diagnostic system according to claim 5,
wherein the processor is configured to:
determine a position and an inclined angle of the third line based on specification information registered in advance.

7. The ultrasound diagnostic system according to claim 6,
wherein as the specification information, position information on an origin in the graphic image and an angle of a reference line that passes through the origin and that is for determining an inclined angle of a line of the graphic image are set based on a positional relationship with a predetermined position in the distal end part.

8. The ultrasound diagnostic system according to claim 1,
wherein the processor is configured to:
determine whether or not the puncture adapter is mounted on the distal end part based on an input by a user.

9. The ultrasound diagnostic system according to claim 1, further comprising:
a laparoscope,
wherein the processor is configured to:
determine whether or not the puncture adapter is mounted on the distal end part based on a captured image from the laparoscope.
